(19) United States
(12) Patent Application Publication
(10) Pub. No.: US 2004/0096866 A1
(43) Pub. Date: May 20, 2004

(54) Method of detecting macromolecular biopolymers by means of an electrode arrangement

(76) Inventors: Franz Hofmann, Munchen (DE); R Johannes Luyken, Munchen (DE)

Correspondence Address: Jeffrey R Stone, Briggs And Morgan, 2200 IDS Center, Minneapolis, MN, 55402, US

(21) Appl. No.: 10/472,168

(22) PCT Filed: Mar. 12, 2002
(86) PCT No.: PCT/DE02/00868

(30) Foreign Application Priority Data
Mar. 20, 2001 (DE) 101 13 550.5

(51) Int. Cl. G01N 33/53; C12Q 1/68

(52) U.S. Cl. 435/6; 435/7.1; 205/777.5

(57) ABSTRACT

The invention relates to a method of detecting macromolecular biopolymers by means of an electrode arrangement that comprises a first and a second electrode. The inventive method is characterized by carrying out a first electrical measurement on the electrodes. In a further step, a solution to be examined, which may contain the macromolecular biopolymers to be detected, is contacted with the electrode arrangement. In another step, the macromolecular biopolymers to be detected that are contained it the solution to be examined are bound to the scavenger molecules on the first and on the second electrode and the electrode arrangement is contacted with a reagent to increase conductivity of the macromolecular biopolymers, said reagent binding to the macromolecular biopolymers and bestowing them with electroconductivity. A second electrical measurement is carried out on the electrodes and the macromolecular biopolymers are detected on the basis of the comparison of the results of the two electrical measurements on the electrodes.

The invention relates to a method of detecting macromolecular biopolymers by means of an electrode arrangement.

[1] to [6] disclose methods of detecting DNA molecules, which comprise using electrodes or particular electrode arrangements for detection.

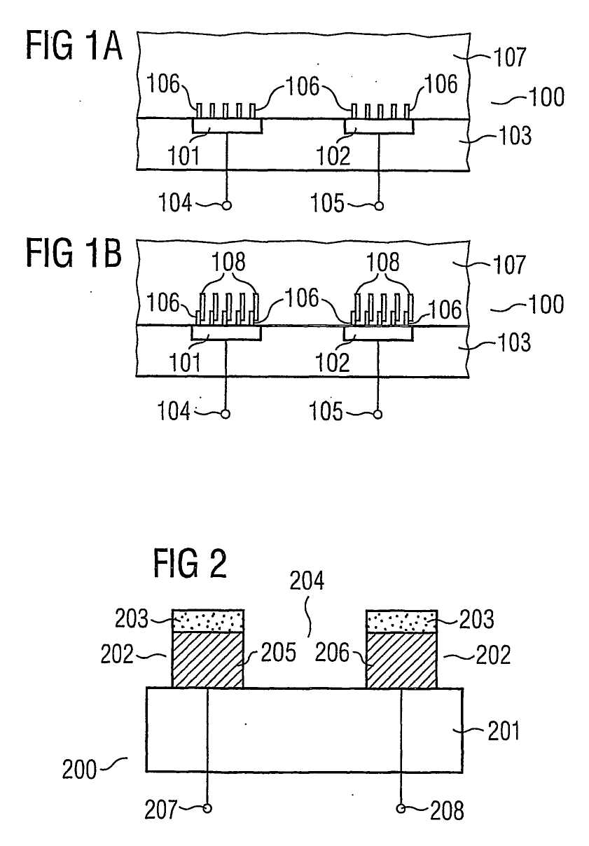

FIG. 1a and FIG. 1b depict a (bio)sensor of the kind described in [2]. The sensor 100 has two electrodes 101 and 102, made of gold, which are embedded in an insulating layer 103 of insulating material. Electrode terminals 104, 105, at which the electric potential applied to the electrode 101, 102 can be tapped, are connected to the electrodes 101, 102. The electrodes 101, 102 are arranged here as planar electrodes. DNA probe molecules 106 are immobilized on each electrode 101, 102 (cf. FIG. 1a). The immobilization was carried out according to the “gold-sulfur coupling”. The analyte to be studied, for example an electrolyte 107, is applied to the electrodes 101, 102.

If the electrolyte 107 contains DNA strands 108 with a sequence which is complementary to the sequence of the DNA probe molecules, then these DNA strands 108 hybridize with the DNA probe molecules 106 (FIG. 1b).

Hybridization between DNA probe molecule 106 and a DNA strand 108 takes place only if the sequences of the particular DNA probe molecules 106 and the corresponding DNA strand 108 are complementary to one another. If this is not the case, then no hybridization takes place. Thus, a DNA probe molecule with a predefined sequence is in each case only capable of binding, i.e. hybridizing, to a particular DNA strand, namely in each case the one with the complementary sequence.

The hybridization causes a change in the capacitance between the electrodes in the sensor described above. This change in capacitance is used as measurement parameter for the detection of DNA molecules.

[7] discloses another procedure of studying the electrolyte with respect to the existence of a DNA strand with predefined sequence. In this procedure, the DNA strands with the sequence of interest are labeled and their existence is determined owing to the reflection properties of the labeled molecules. To this end, the electrolyte is illuminated with light in the visible wavelength range, and the light reflected by the electrolyte, in particular by the DNA strand to be detected, is detected. Owing to the reflection behavior, i.e. in particular owing to the reflected beams of light detected, it is determined whether or not the electrolyte contains the DNA strand with the correspondingly predefined sequence, which is to be determined.

This procedure is very complicated, since it requires very good knowledge of the reflection behavior of the relevant labeled DNA strand and furthermore necessitates labeling of the DNA strands before the start of the process.

Furthermore, the means of detection for detecting the reflected beams of light need to be adjusted very accurately, in order to be able to detect the reflected beams of light at all.

Thus said procedure is expensive, complicated and very sensitive to interfering influences, and this can very readily distort the result of the measurement.

It is furthermore known from affinity chromatography (cf. [8]) to use immobilized low molecular weight molecules, in particular ligands of high specificity and affinity, in order to specifically bind peptides and proteins, for example enzymes, in the analyte.

Finally, [9] discloses using DNA as template for formation of a conducting silver wire between two electrodes by localizing silver ions on the DNA which are then reduced to metallic silver. This method is also described in [11].

Furthermore, [12] discloses a method and a device for identifying a biopolymer sequence on solid surfaces. Here, a first biopolymer applied to a solid substrate is contacted with a second biopolymer with affinity therefor.

Moreover, [13] discloses an affinity sensor for detecting specific molecular binding events, which is to be applied, in particular, to DNA microarray assays, for example.

The above detection methods disclosed in [1] to [8] have the disadvantage of requiring relatively large amounts of micromolecular biopolymers to be detected, i.e. their detection sensitivity is relatively low.

The present invention has for its object to provide an alternative method of detecting macromolecular biopolymers which has a simple design and high detection sensitivity.

This object is achieved by the method having the features as claimed in the independent patent claim.

A method of detecting macromolecular biopolymers uses an electrode arrangement which has a first and a second electrode.

Both the first electrode and the second electrode are provided with scavenger molecules which can bind macromolecular biopolymers. These scavenger molecules may be molecules of a single kind or else molecules of a first and a second kind, i.e. different kinds of scavenger molecules.

The method further comprises carrying out a first electrical measurement at the electrodes. In a further step, a solution to be studied which may contain the macromolecular biopolymers to be detected is contacted with the electrode arrangement. In a further step, macromolecular biopolymers to be detected which are present in the solution to be studied are bound to the scavenger molecules on the first and the second electrode. The electrode arrangement is further contacted with a reagent for increasing the conductivity of macromolecular biopolymers, which binds to said macromolecular biopolymers and imparts to these increased electric conductivity. Subsequently, a second electrical measurement at the electrodes is carried out and the macromolecular biopolymers are detected depending on comparison of the results of the two electrical measurements at the electrodes.

Graphically expressed, the present method is based on the finding that macromolecular biopolymers which are generally nonconductive or only poorly conductive can be rendered electrically conductive by attaching/binding a reagent which increases the conductivity of the biopolymers and the now conductive macromolecular biopolymers to be detected are used as a “conductivity bridge” between two electrodes, which influences the current flow between the electrodes. Due to the fact that the conductivity bridge which may also be understood as being a “molecular shortcut” between two electrodes can be formed in principle only by a single molecule, the present method has a higher detection sensitivity than the known methods, namely a sensitivity of a single molecule of the macromolecular biopolymers to be detected.

Owing to the above-described principle, preference is given to determining resistance or current flow in the electrical measurement at the electrodes.

The scavenger molecule used in the method described herein may be a single type of molecule, for example a double-stranded nucleic acid with a defined nucleic acid sequence. In a development of the invention, however, the scavenger molecules are at least first and second scavenger molecules, for example at least two oligonucleotides with different nucleic acid sequences (which therefore have different binding specificities) or two antibodies which can bind different surface regions (epitopes) of a macromolecular biopolymer.

Detecting means in accordance with the invention both qualitative and quantitative detection of macromolecular biopolymers in an analyte to be studied, meaning that the term “detecting” also includes determining the absence of macromolecular biopolymers in the analyte.

A reagent for increasing the conductivity of macromolecular biopolymers here means a reagent capable of binding, preferably specifically, to macromolecular biopolymers and having a conductivity for electric current, which is higher than that of the macromolecular biopolymers to be detected.

A reagent of this kind for increasing conductivity is preferably a reagent which can be reduced in the chemical sense, i.e. a reagent capable of donating electrodes, resulting in a decrease of the oxidation state of at least one of the atoms of said reagent. In this case, the reagent preferably contains metal ions which can not only be chemically reduced and bind to macromolecular biopolymers but also be readily dissolved in solvents suitable for macromolecular biopolymers. Examples of such metal ions are silver, gold, copper or nickel ions or mixtures thereof which can bind as cations to negatively charged groups on the surface of macromolecular biopolymers by electrostatic interaction. If the biopolymers to be detected are nucleic acids, such cations are bound to the negatively charged phosphate backbone of said nucleic acids. If proteins are to be detected, such cations can bind via the side chains of acidic amino acids such as aspartate or glutamate.

Other types of the reagents for increasing conductivity are soluble polymers or oligomers conducting the electric current which are positively charged in the conducting state. Examples of such reagents are suitably substituted polypyrroles, polythiophenes or oligothiophenes (having, for example, from 2 to 10 thiophene units, for example 6 thiophene units). Examples of substituents mediating the solubility of these polymers or oligomers in solvents compatible with macromolecular biopolymers, preferably in aqueous media, are sulfonic acid or carboxylic acid groups linked to the aromatic backbone via alkylene units. In the case of polypyrroles, substitution is preferably via the 3-position of the aromatic ring. These reagents preferably attach to the macromolecular biopolymers to be detected via electrostatic interactions with charged groups or residues of said biopolymers. If the biopolymers to be detected are nucleic acids, however, it is also conceivable that the reagent for increasing conductivity may also bond through interactions with other regions of the nucleic acid, such as, for example, the minor groove of nucleic acids.

When using a reduceable reagent such as, for example, silver ions, the electrode arrangement in the method described herein is contacted with a reducing agent which reduces the reagent for increasing conductivity (which is bound to the macromolecular biopolymers). The reduction may be carried out using known and common organic or inorganic reducing agents such as, for example, hydroquinone and hydrogen sulfite. If, on the other hand, the abovementioned conducting polymers or oligomers are used, a chemical reduction of the reagent for increasing conductivity is not necessary, since the reagent in its bondable form already conducts electric current.

At this point it should be noted that in the method disclosed herein it is not only possible to contact the electrode arrangement with the reagent for increasing the conductivity of macromolecular biopolymers after immobilization of the macromolecular biopolymers to be detected. Rather, it is also possible first to contact the solution to be studied with the reagent for increasing conductivity and then to bind, i.e. to immobilize, the biopolymers to be detected to the electrodes.

Macromolecular biopolymers which may be detected by the method are nucleic acids, oligonucleotides, proteins, peptides or complexes thereof, i.e. complexes of nucleic acids and proteins, for example.

Specifically, macromolecular biopolymers here means on the one hand nucleic acids such as DNA and RNA molecules or else smaller nucleic acid molecules such as oligonucleotides of, for example, approx. 10 to 40 base pairs (bp) in length. The nucleic acids may be double-stranded but may also have at least single-stranded regions or may be present completely in the form of single strands, for example due to previous thermal denaturation or another kind of strand separation for their detection. The sequence of the nucleic acids to be detected may be at least partially or else completely predefined, i.e. known. Other macromolecular biopolymers which may be detected here are proteins or peptides. These may be composed of the 20 amino acids usually found in proteins but may also contain not naturally occurring amino acids or may be modified, for example by sugar residues (oligosaccharides) or contain posttranslational modifications. It is furthermore also possible to detect complexes of nucleic acids and proteins, as formed, for example, by a (specifically) DNA-binding protein such as a translation factor with a DNA molecule having the corresponding recognition sequence.

If the macromolecular biopolymers to be detected are proteins or peptides, then the scavenger molecules (located on the electrodes) are preferred ligands with a binding activity for the proteins or peptides to be detected. This makes it possible for the proteins or peptides to be detected to bind to the electrodes on which the corresponding ligands are arranged. The scavenger molecules/ligands are themselves preferably linked to the electrodes via covalent bonds.

Suitable ligands for proteins and peptides are low molecular weight enzyme agonists or enzyme antagonists, pharmaceuticals, sugars or antibodies or any molecule capable of specifically binding proteins or peptides.

When nucleic acids or oligonucleotides are detected by the method described herein, they may be present both in single-stranded and in double-stranded form. Preferred scavenger molecules used for nucleic acids are DNA probe molecules, which is why the nucleic acids then have at least one single-stranded region accessible to hybridization. Preference is given to using DNA probe molecules with a sequence (fully) complementary to the single-stranded region. The DNA probe molecules here may be oligonucleotides or else have longer nucleotide sequences, as long as the latter do not form any of the intermolecular structures which prevent the probe molecule from hybridizing with the nucleic acid to be detected. It is, however, also possible to use DNA- or RNA-binding proteins or agents as scavenger molecules.

The fact that the biopolymers to be detected are usually not identical in any region of their secondary and/or tertiary structure is a problem when detecting macromolecular biopolymers. Thus, for example, polypeptides and proteins have in principle a different and unique spatial structure at each location/in each region (of the surface). Nucleic acids to be detected normally have a different base sequence at their two termini (i.e. the 3′ terminus and the 5′ terminus).

To solve this problem, one embodiment of the method described herein uses at least first and second scavenger molecules. In this case, the first scavenger molecules are capable of binding (specifically) a first region of a biopolymer to be detected and the second scavenger molecules are capable of binding (specifically) a second region of a biopolymer to be detected. This ensures formation of the conductivity bridge described herein.

The term “region of a macromolecular biopolymer to be detected” means in accordance with the invention a region which either has a special three-dimensional (spatial) structure, as is the case for proteins, or has, as is the case for nucleic acids which can, in principle, adopt an identical or very similar three-dimensional structure, a nucleotide sequence differing from the other regions. Consequently, the scavenger molecules may be, for example, two antibodies which recognize in each case a special epitope of the protein to be detected or may be an antibody which recognizes an epitope on the protein to be detected and a peptide which binds into the (spatially distant) active site of said protein, or may be two oligonucleotides with a particular sequence which is in each case complementary to the nucleotide sequence of either of the two termini.

In this embodiment of the invention, preference is given to applying the at least first and second scavenger molecules in each case in a homogeneous distribution, i.e. in a uniform distribution, to the two electrodes. As a result of this, the macromolecular biopolymers are bound to the electrodes by means of the scavenger molecules independently of the orientation of said biopolymers in the solution to be detected. Said uniform distribution may be achieved, for example, by first preparing a mixture of the scavenger molecules and then applying said mixture to the electrodes.

The present method may be carried out in principle by using any electrode arrangement known in the field of biosensing. The electrode arrangement may comprise, for example, a usual substrate which has, for example, silicon or gallium arsenide and to which firstly a gold layer and a silicon nitride layer have been applied and which has then been structured by means of conventional lithographic and etching techniques to generate the electrode arrangement(s).

In structuring, the distance between the two electrodes may vary, depending on the kind of structuring technique used and the type of macromolecules to be detected. The distance between the electrodes is generally from approx. 5 nanometers (nm) to 100 or several 100 nanometers. Shorter distances in the range from approx. 5 nm to approx. 30 or 40 nm are preferred here for detecting smaller (shorter) biopolymers, even though it is currently technologically more complicated to generate such distances, for example by means of lithographic or doping methods, than distances in the range from approx. 100 nm to several 100 nm.

If the three-dimensional structure of the macromolecular biopolymers to be detected is known, it is possible to estimate the preferred electrode distance to be used on the basis of the size of said biopolymer. For example, in the case of nucleic acids whose 3D structure is generally known, it can be assumed by way of approximation, starting from the known helical pitch of ideal A, B and Z DNA (cf. [10]), which is, for example, for B DNA 0.34 nm per helical turn and base pair, that 10 base pairs (bp) bridge a distance of 3.4 nm and thus the distance between the electrodes can be estimated.

In order to detect a nucleic acid which is 50 bp, corresponding to approx. 17 nm, in length, using oligonucleotides as scavenger molecules which have in each case a complementary sequence of 15 bp with a total length of in each case 20 bp, the distance between the electrodes should be approx. 17 nm+2□5□0.34=approx. 21.4 nm. It should be noted here that adjusting the distance between the electrodes by structuring is not required for detecting a biopolymer mentioned herein. Rather, it is also possible, for example, to change the distance to be bridged by the macromolecular biopolymers to be detected and thus the distance between the electrodes by varying the length of the scavenger molecules. Thus the scavenger molecules may, where appropriate, be extended or truncated. If the scavenger molecules used are nucleic acids or oligonucleotides, it would be appropriate, for example, to extend said scavenger molecules by additional nucleotides, since they (said scavenger molecules) too can bind conductive reagents such as metal cations to essentially the same extent as biopolymers to be detected. It is therefore possible to determine in the method of the invention the (optimal) distance between the electrodes purely empirically without knowledge of the three-dimensional extension of the macromolecular biopolymer to be detected.

In this connection, it should be noted that it is also possible to use scavenger molecules which per se do not have sufficient conductivity but which can be rendered conductive by modifications. In the case of a hormone as actual scavenger molecule, for example, it is possible to use a negatively charged spacer for binding the hormone to the electrodes.

It is of course possible, with the aid of the method disclosed herein, to detect not just a single type of biopolymers in an individual series of measurements. Rather, it is possible to detect a plurality of macromolecular biopolymers simultaneously or else successively. For this purpose, it is merely necessary to use a substrate which has a plurality of electrode arrangements with in each case two electrodes, i.e. an electrode pair. The in each case different scavenger molecules, each of which has a “specific” binding affinity for a particular biopolymer to be detected, are then bound on each of said electrode pairs. It is also possible to use a plurality of electrode pairs, each pair being provided only with one scavenger molecule or at least first and second scavenger molecules, which binds specifically one of the biopolymers to be detected.

An example of an electrode arrangement which may be used for carrying out the method described herein is a conventional interdigitated electrode. Consequently, a biosensor provided with a plurality of interdigitated electrodes, i.e. an electrode array, can be employed for parallel or multiple determinations. Another usable electrode arrangement is an electrode arrangement in the form of a trench or a cavity, which is formed, for example, by holding regions such as, for example, a gold layer on which the scavenger molecules capable of binding the macromolecular biopolymers are immobilized being located on two opposite side walls.

The present method comprises as first method step carrying out a first electrical measurement at the electrodes. For this first measurement, the scavenger molecules may have already be applied to the means for immobilization, but they need not be. The scavenger molecules can be applied by any technique known for this purpose. If multiple determinations are to be carried out the scavenger molecules may be applied, for example, with the aid of inkjet printing techniques.

A medium, for example an electrolyte, is contacted with the electrode arrangement. This is done in such a way that the macromolecular biopolymers can bind to the scavenger molecules. If the medium contains a plurality of macromolecular biopolymers to be detected, the conditions are chosen so that said biopolymers can bind in each case either simultaneously or successively to their corresponding scavenger molecules.

After waiting a sufficient period of time for the macromolecular biopolymers to bind to the corresponding scavenger molecule(s), unbound scavenger molecules may be removed from the electrodes on which they are located.

If the scavenger molecules are nucleic acid (DNA) strands, this is carried out, for example, enzymatically by means of an enzyme which selectively breaks down single-stranded DNA. Here, the selectivity of the degrading enzyme for single-stranded DNA needs to be taken into account. If the enzyme selected for breaking down non-hybridized DNA single strands does not have this selectivity, then the hybridized double-stranded DNA to be detected may also undesirably be broken down.

The unbound DNA probe molecules may be removed from the particular electrode by using, in particular, DNA nucleases, for example a nuclease from mung beans, the nuclease P1 or the nuclease S1. It is likewise possible to use DNA polymerases which, due to their 5′□3 exonuclease activity or their 3′□5′ exonuclease activity, are capable of breaking down single-stranded DNA.

If the scavenger molecules are low molecular weight ligands, the latter, if unbound, can also be removed enzymatically.

To this end, the ligands are covalently linked to the electrodes via an enzymatically cleavable linkage, for example via an ester linkage.

In this case, it is possible to use, for example, a carboxyl ester hydrolase (esterase) in order to remove unbound ligand molecules. This enzyme hydrolyzes that ester linkage between the electrode and the particular ligand molecule which has not been bound by a peptide or protein. In contrast, the ester linkages between the electrode and those molecules which have interacted with peptides or proteins by way of bonding remain intact, owing to reduced sterical accessibility which is caused by the space-filling properties of the bound peptide or protein.

Removing the unbound scavenger molecules is optional but may be advantageous in that the measured signal obtained is not influenced by, for example, scavenger molecules which are (like oligonucleotides) likewise capable of binding reagents for increasing the conductivity of the macromolecular biopolymers, such as reducible metal cations.

The electrode arrangement is contacted, either before or after removing unbound scavenger molecules, with a reagent for increasing the conductivity of macromolecular biopolymers, which binds to said macromolecular biopolymers and imparts to these electrical conductivity. Here too, the reagent is given sufficient time to bind to the macromolecular biopolymers.

If the reagent is still present in a form which does not yet increase the conductivity of the macromolecular biopolymers to the desired extent (as is the case for metal cations such as Ag⁺ or Au⁺), it is possible to convert this not yet sufficiently conductive form in another method step to such a conductive form (e.g. metallic silver or gold).

Subsequently, a second electrical measurement is carried out at the electrodes. The values determined by the first and the second electrical measurement are then compared with one another. If the measured values of the measurement parameter used differ in such a way that the difference between the values determined is greater than a predefined threshold, it is assumed that macromolecular biopolymers have bound to scavenger molecules or, generally, to the electrodes, causing the change in intensity of the signal received at the receiver.

If the difference between the values of the first and second electrical measurement is greater than the predefined threshold, the stated result is that the relevant macromolecular biopolymers which specifically bind a scavenger molecule have been bound and thus the medium contained the relevant macromolecular biopolymers.

In this way, macromolecular biopolymers have been detected.

The method may be designed in such a way that a reference measurement and a measurement for detecting macromolecular biopolymers are carried out simultaneously. This may be done, for example, by carrying out a reference measurement only with the medium and, at the same time, a measurement with the medium containing (or else not containing) the macromolecular biopolymers to be detected, if qualitative detection is desired, for example.

Exemplary embodiments of the invention are illustrated in more detail hereinbelow and are depicted in the figures in which

FIGS. 1a and 1b depict a sketch of planar electrodes by means of which the existance (FIG. 1a) or non-existance (FIG. 1b) of DNA strands to be detected in an electrolyte can be detected;

FIG. 2 depicts a sketch of an electrode arrangement which can be used for carrying out the method of the invention;

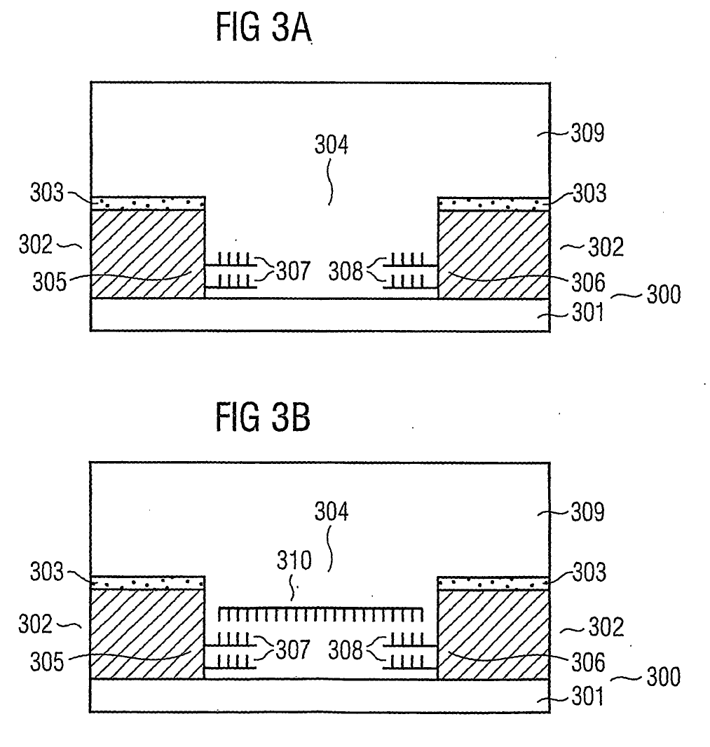

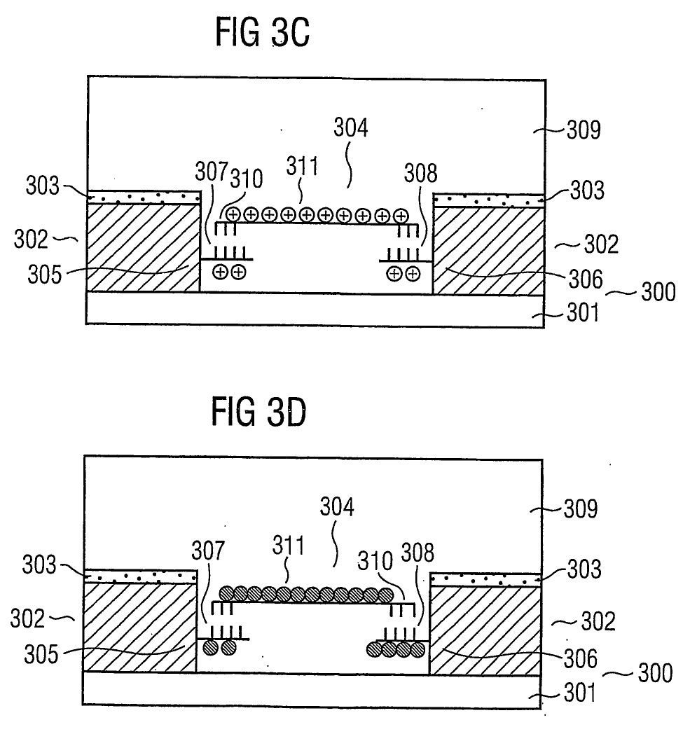

FIGS. 3a to 3d depict different method states of a method of detecting nucleic acids according to an exemplary embodiment of the invention;

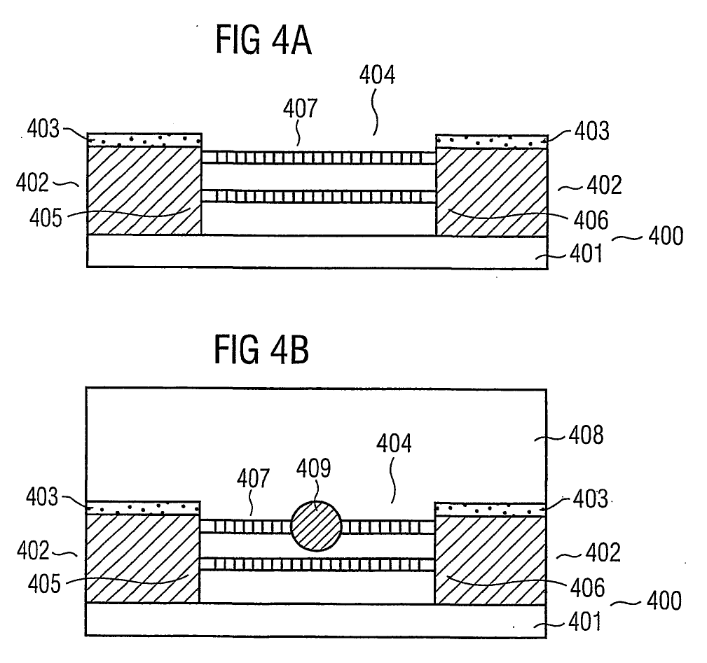

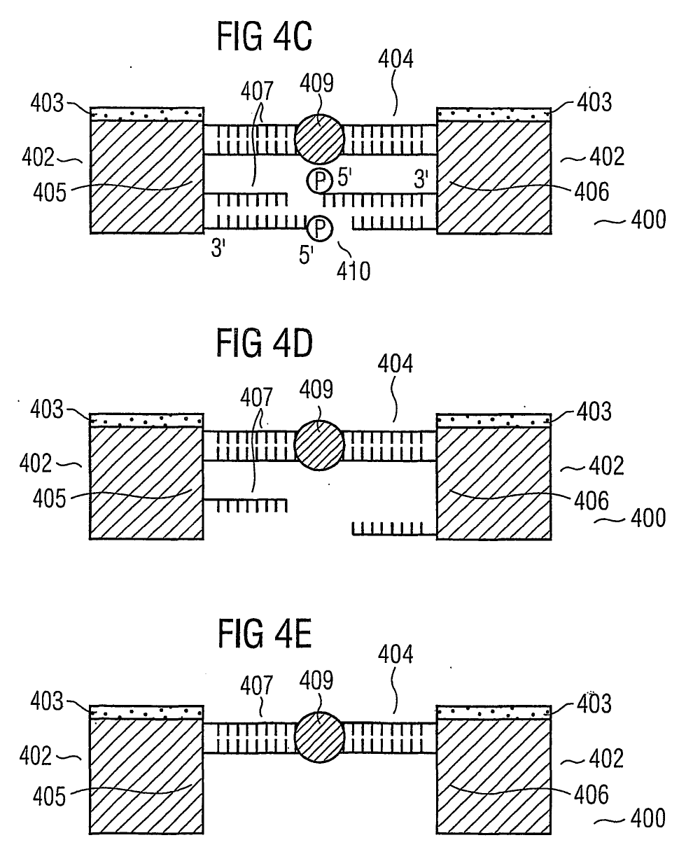

FIGS. 4a to 4e depict different method states of a method of detecting proteins according to another exemplary embodiment of the invention.

FIG. 2 depicts a sectional view of a trench-shaped electrode arrangement 200 which may be used for the methods disclosed herein. In this electrode arrangement 200, a gold layer 202 and a silicon nitride layer 203 are applied to an insulating substrate 201, for example a silicon oxide substrate. Structuring, for example by means of a common chemical etching process, produces the trench shape 204, the electrode pair of the first electrode and the second electrode being formed by the opposite side walls 205 and 206. The first electrode 205 is provided with a first electrical terminal 207 and the second electrode is provided with a second electrical terminal 208. In this connection, it should be mentioned that a sensor suitable for multiple measurements may have, for example, a plurality of trenches arranged in parallel.

FIG. 3a depicts a section of an electrode arrangement 300 with an insulating substrate 301, a first layer 302, a silicon nitride layer 303, a first electrode 305 and a second electrode 306, said first electrode 305 and said second electrode 306 being made of gold. The electrode arrangement forms a trench 304.

Alternatively, the electrodes 305 and 306 may also be made of silicon oxide and may be coated with a material suitable for immobilizing the scavenger molecules thereupon.

It is possible, for example, to use known alkoxysilane derivatives such as

3-glycidoxypropylmethyloxysilane,

3-acetoxypropyltrimethoxysilane,

3-aminopropyltriethoxysilane,

4-(hydroxybutyramido)propyltriethoxysilane,

3-N,N-bis(2-hydroxyethyl)aminopropyltriethoxysilane, or other related materials which are capable of covalently binding with their one end to the silicon oxide surface and providing with their other end the probe molecule to be immobilized with a chemically reactive group such as an epoxy, acetoxy, amine or hydroxyl radical for reaction.

If a scavenger molecule to be immobilized reacts with an activated group of this kind, it is immobilized via the chosen material as a kind of covalent linker on the surface of the coating on the electrode.

DNA probe molecules 307, 308 are applied as scavenger molecules to the immobilized regions of the electrodes 305, 306. In the case of the gold electrodes shown here, immobilization is carried out, for example, via gold-sulfur coupling.

Primary DNA probe molecules 307 are applied to the first electrode 305, their nucleotide sequence being complementary to, a predefined first DNA sequence of a nucleic acid to be detected. Secondary DNA probe molecules 308 are applied to the second electrode 306, their nucleotide sequence being complementary to a predefined second DNA sequence of the nucleic acid to be detected. This embodiment is thus an example of using first and second scavenger molecules with different specificity.

A first electrical measurement is carried out at the electrodes either before or after immobilization of the DNA probe molecules. This involves determining preferably the resistance or current flow by means of two electrode terminals, not shown in FIG. 3, at the first and second electrode 305, 306 and a connected measuring device (likewise not shown). In the first electrical measurement, a reference value, for example for resistance, is determined and stored in a storage device (not shown).

DNA strand sequences complementary in each case to the probe molecule sequences can hybridize to the pyrimidine bases adenine (A), guanine (G), thymine (T) or cytosine (C), in the usual way, i.e. by base pairing via hydrogen bonds between A and T and, respectively, between C and G.

FIG. 3a further depicts an electrolyte 309 which is contacted with the electrodes 305, 306 and the DNA probe molecules 307, 308.

FIG. 3b depicts the electrode arrangement 300 in the case that the electrolyte 309 contains a DNA molecule 310 which has a predefined first sequence and a predefined second sequence which are in each case complementary to the sequence of the first DNA probe molecule 307 and the second DNA molecule 308, respectively. The DNA molecule may be single-stranded, as indicated in FIG. 3, or double-stranded.

Owing to the sequence specificity of the base pairing, the DNA strand 310 to be detected (the DNA molecule to be detected) hybridizes in this case to the primary DNA probe molecule 307 via the first predefined sequence and to the secondary DNA probe molecule 308 via the second predefined sequence. Hybridization may be spontaneous but, in the case of double-stranded nucleic acid molecules 310, may also be caused, for example, by thermal denaturation or induction of fluidic movement perpendicular to the electrodes, as described in [9].

As FIG. 3b shows, hybridization results in the formation of a DNA “bridge” between the electrodes.

In an optional step, hydrolyzing of non-hybridized single-stranded DNA probe molecules 307 or 308 (cf. FIG. 3b) is caused by means of a biochemical method, for example by means of adding DNA nucleases to the electrolyte 309. If single-stranded DNA is to be detected, this step should be dispensed with, however, if said step causes the possibility of the single strand 310 to be detected likewise being broken down.

When removing non-hybridized scavenger molecules, the selectivity of the degrading enzyme for single-stranded DNA needs to be taken into account. If the enzyme selected for breaking down non-hybridized DNA single strands does not have said selectivity, then it is possible that the hybridized double-strand DNA to be detected is also undesirably broken down, leading to a distortion of the result of the measurement.

After removing the single-strand DNA probe molecules, i.e. the primary DNA probe molecules 307 on the first electrode 305 and the secondary DNA probe molecules 308 on the second electrode 102, only the DNA strands 307 and 308 hybridized with the DNA molecule to be detected are present (cf. FIG. 3c).

The single-strand DNA probe molecules 306 and 307 on the two electrodes may be removed by adding, for example, any of the following substances:

nuclease from mung beans,

nuclease P1, or

nuclease S1.

DNA polymerases which, due to their 5′□3′ exonuclease activity or their 3′□5′ exonuclease activity, are capable of breaking down single-stranded DNA may also be used for this purpose.

After or else, where appropriate, prior to this degradation step, the electrode arrangement 300 is contacted with a reagent for increasing the conductivity of macromolecular biopolymers, which binds to said macromolecular biopolymers and imparts to these electric conductivity. Examples of this reagent are silver ions 311 dissolved in alkaline medium, as described in [9]. The resulting binding of the silver ions 311 to the DNA molecules, depicted in FIG. 3c, takes place via replacing the sodium ions bound to the phosphate backbone.

The conductivity bridge is formed by finally reducing the silver ions 311 bound to the DNA molecules. For this purpose it is possible, as described in [9] first to form small silver aggregates on the DNA by means of a basic hydroquinone solution and then to convert the DNA to a “wire” completely covered with metallic silver by adding an acidic “developer solution” of hydroquinone and silver ions. Such a “wire” is depicted in FIG. 3d.

Using the abovementioned electrode terminals, not shown, and the connected measuring device (likewise not shown), a second electrical measurement, for example a second resistance measurement, is then carried out according to this first exemplary embodiment. By means of the second resistance measurement, a resistance value is determined which is compared with the reference value.

If the difference between these resistances is greater than a predefined threshold, then this means that the electrolyte 309 contained a DNA strand.

In this case, the user of the measuring device receives a corresponding output signal from said measuring device.

FIG. 4 depicts another embodiment of the present method, in which a protein, more specifically a DNA-binding protein such as, for example, a transcription factor, is detected as biopolymer to be detected with the aid of the electrode arrangement 400. Said electrode arrangement 400 has an insulating substrate 401, a first layer 402, a silicon nitride layer 403, a first electrode 405 and a second electrode 406. The first electrode 405 and the second electrode 406 are again made of gold. The electrode arrangement likewise forms a trench 304.

This embodiment uses only a single type of scavenger molecule, namely double-stranded nucleic acid molecules 407 which have a recognition sequence for the DNA-binding protein (FIG. 4a).

The nucleic acid molecules 407 are immobilized on the two electrodes 405 and 406 via gold-sulfur coupling. For this purpose, thiol groups are attached in each case to the 3′ termini of the nucleic acid 407, this being possible, for example as described in [9], via enzymic extension of said nucleic acid 407 with oligonucleotides having disulfide groups at the 3′ end (cf. FIG. 3).

Alternatively, however, it is also possible to bind the nucleic acid molecule 407 itself, which serves as scavenger molecule, to the two electrodes 405 and 406 via a first and a second oligonucleotide attached to the first electrode 405 and, respectively, to the second electrode 406, i.e. via two further scavenger molecules.

This embodiment likewise comprises carrying out a first electrical measurement at the electrodes, either before or after immobilization of the DNA probe molecules 407, preferably determining the resistance or current flow by means of two electrode terminals, not depicted in FIG. 4, at the first and second electrode 405 and 406 and a connected measuring device (likewise not shown), and then determining in the first electrical measurement a reference value, for example for resistance, and storing said value in a storage device (not shown).

Subsequently, an electrolyte 408 is contacted with the electrodes 405 and 406 and the DNA probe molecules 407. FIG. 4b depicts the case where the electrolyte 408 contains a protein molecule to be detected 409. In this case, the protein 409 binds to its recognition sequence located on the scavenger molecule 407.

In a further method step, the two electrodes 405, 406 and the scavenger molecules 407 (both those scavenger molecules which have formed a complex with a protein molecule to be detected 409 and the uncomplexed molecules) are contacted with a biochemical reagent such as a restriction endonuclease which has a specific recognition sequence, i.e. a specific restriction cleavage site. Preference is given to using a restriction endonuclease for which the nucleic acid molecules 407 possess a unique restriction cleavage site which is located in that region of the nucleic acid molecules 407 to which the protein to be detected 409 has bound. The spatial shielding by the protein 409 causes only those DNA molecules (scavenger molecules) 407 to be cleaved by the restriction endonuclease which do not have bound a protein molecule 409. In contrast, DNA molecules 407 which have formed a complex with a protein molecule to be detected 409 remain intact, i.e. they are not cleaved (cf. FIG. 4b).

After treatment with the restriction endonuclease, i.e. after double-strand cleavage, uncomplexed scavenger molecules 407 have protruding, free ends 410 with a 5′ phosphate group, as FIG. 4c diagrammatically shows.

In a further biochemical method step, the scavenger molecules 407 are treated with an enzyme such as lamda (●) exonuclease which selectively digests/breaks down an individual strand of a double-stranded DNA duplex from its 5′-phosphorylated end. In the case of the cleaved scavenger molecules 407 which have such a phosphorylated end 410, this means that in each case this strand is broken down and thus the complementary, no longer hybridized single strand of the scavenger molecule 407 is retained (FIG. 4d).

Said single strand can be removed in a further biochemical method step, it being possible to use suitable single-strand-specific nucleases such as nuclease P1 mentioned in the exemplary embodiment described on the basis of FIG. 3. As a result, this treatment leaves only scavenger molecules 407 to which the protein to be detected 409 is bound or no scavenger molecules 407, if, for example, no such protein was present in the electrolyte 409 (FIG. 4e).

Subsequently it is possible, similarly to the procedure described in the previous exemplary embodiment, to contact the electrode arrangement 400 with a reagent for increasing the conductivity of macromolecular biopolymers, such as silver ions dissolved in alkaline medium. These are, after their binding to the complex of scavenger molecule 407 and protein to be detected 409, reduced to form a conductivity bridge, as likewise described above (cf. FIGS. 3d, 3e).

Finally, a second electrical measurement using electrode terminals, not shown, and the connected measuring device (likewise not shown) is carried out, and the presence or absence of the protein to be detected is inferred from comparison of the measured value obtained.

It is obvious that the method according to this second exemplary embodiment can detect not only proteins such as a DNA-binding protein but also complexes of macromolecular biopolymers such as nucleic acid/protein complexes.

1. A method of detecting macromolecular biopolymers by means of an electrode arrangement having:
a first electrode,
a second electrode,
a) in which method the first electrode is provided with scavenger molecules which can bind macromolecular biopolymers, and in which method the second electrode is provided with scavenger molecules capable of binding macromolecular biopolymers, said scavenger molecules being at least first and second scavenger molecules, said first scavenger molecules being capable of binding a first region of a biopolymer to be detected and said second scavenger molecules being capable of binding a second region of a biopolymer to be detected, said first and said second electrode being provided in each case with first and second scavenger molecules,
b) in which method a solution to be studied is contacted with the electrode arrangement, it being possible for said solution to contain the macromolecular biopolymers to be detected,
c) in which method macromolecular biopolymers to be detected which are contained in the solution to be studied are bound to the scavenger molecules on the first and the second electrode,
d) in which method the electrode arrangement is contacted with a reagent for increasing the conductivity of macromolecular biopolymers, which binds to said macromolecular biopolymers and imparts to them increased electric conductivity,
e) in which method subsequently a electrical measurement is carried out on the electrodes,
f) in which method the macromolecular biopolymers are detected, depending on comparison of the results of the two electrical measurements at the electrodes.

2. The method as claimed in claim 1, wherein the reagent for increasing the conductivity of macromolecular biopolymers is (chemically) reduceable.

3. The method as claimed in claim 2, wherein the reagent for increasing conductivity contains metal ions.

4. The method as claimed in claim 3, wherein the metal ions are selected from the group consisting of silver, gold, copper, nickel ions and mixtures thereof.

5. The method as claimed in any of claims 2 to 4, wherein step d) is followed by contacting the electrode arrangement with a reducing agent which reduces the reagent for increasing conductivity.

6. The method as claimed in any of the preceding claims, wherein the macromolecular biopolymers used are nucleic acids, oligonucleotides, proteins, peptides or complexes thereof.

7. The method as claimed in any of the preceding claims, wherein the scavenger molecules are capable of specifically binding the macromolecular biopolymers.

8. The method as claimed in claim 7, in which the first and second scavenger molecules are in each case applied to the two electrodes in a homogeneously distributed manner.

9. The method as claimed in any of claims 6 to 8, in which the nucleic acids detected are DNA or RNA molecules.

10. The method as claimed in claim 9, in which the DNA or RNA molecules of a predefined sequence are detected.

11. The method as claimed in claim 10, in which the DNA or RNA molecules to be detected have at least one single-stranded region.

12. The method as claimed in claim 11, in which the scavenger molecules used are DNA probe molecules having a sequence complementary to the single-stranded region.

13. The method as claimed in claim 12, in which unbound DNA probe molecules are removed from the electrodes by contacting an enzyme with nuclease activity with the two electrodes.

14. The method as claimed in claim 13, in which the enzyme with nuclease activity used is at least one of the following enzymes:
a) nuclease from mung beans
b) nuclease P1
c) nuclease S1, or
d) DNA polymerases which due to their 5′□3′ exonuclease activity or their 3′□5′ exonuclease activity or their exonuclease activity, are capable of breaking down single-stranded DNA.

15. The method as claimed in any of claims 6 to 8, in which the scavenger molecules used are ligands capable of specifically binding proteins or peptides.

16. The method as claimed in claim 15, wherein unbound ligands are released from the two electrodes by contacting a material with said two electrodes which is capable of hydrolyzing the chemical linkage between the ligands and the electrodes.

17. The method as claimed in claim 16, wherein the material being contacted with the electrodes is an enzyme.

18. The method as claimed in claim 17, wherein the enzyme being contacted with the electrodes is a carboxyl ester hydrolase (esterase).
